(19) United States
(12) Patent Application Publication
(10) Pub. No.: US 2024/0168676 A1
(43) Pub. Date: May 23, 2024

(54) GENERATING AND UPDATING SOFT INFORMATION FOR DNA-BASED STORAGE SYSTEMS

(71) Applicant: Western Digital Technologies, Inc., San Jose, CA (US)

(72) Inventors: David Avraham, Even Yehuda (IL); Ran Zamir, Ramat Gan (IL); Alexander Bazarsky, Holon (IL)

(21) Appl. No.: 18/356,110

(22) Filed: Jul. 20, 2023

Related U.S. Application Data
(60) Provisional application No. 63/427,621, filed on Nov. 23, 2022.

(51) Int. Cl. G06F 3/06; G16B 50/30

(52) U.S. Cl. CPC G06F 3/0655; G06F 3/0619; G06F 3/0673; G16B 50/30

(57) ABSTRACT

A DNA-based storage system generates soft information that increases an efficiency and/or a reliability of a low-density parity-check (LDPC) decoder of the DNA-based storage system. The soft information is generated by comparing corresponding DNA segments of multiple copies of a DNA sequence to determine a ratio of DNA symbols in agreement in each of the DNA segments and/or whether a length of each of the DNA segments are the same. The ratio of symbols in agreement and/or the length information may be used to determine a bit error rate (BER) of a particular DNA segment. The BER of each DNA segment may then be used to determine soft information, or a log likelihood ratio (LLR), associated with that particular DNA segment.

### CROSS-REFERENCE TO RELATED APPLICATIONS

The present application claims priority to U.S. Provisional Application 63/427,621 entitled “GENERATING AND UPDATING SOFT INFORMATION FOR DNA-BASED STORAGE SYSTEMS”, filed Nov. 23, 2022, the entire disclosure of which is hereby incorporated by reference in its entirety.

### BACKGROUND

DNA-based storage systems are emerging as a promising storage technology. DNA is a long molecule made up of four nucleotide bases—adenine (A), cytosine (C), thymine (T) and guanine (G). For storage purposes, base units (ACTG) of synthesized DNA can be used to encode information—similar to how a string of ones and zeros represents data in traditional electronic storage systems. The encoded information may then be stored, subsequently accessed and decoded.

For example, DNA-based storage systems typically store DNA data using three main processes—synthesis (or writing) in which the base units of synthesized DNA are joined together to produce a desired DNA string; storage, in which the DNA string is stored in a DNA-based storage medium; and sequencing (or reading), in which the DNA string is translated to binary/digital data.

While DNA-based storage systems are more dense than traditional electronic data storage systems, DNA-based storage systems are more prone to errors. For example, during synthesis, storage and/or sequencing, various symbols in the DNA string may be inserted or deleted. In other examples, during synthesis, storage and/or sequencing, one symbol (or multiple symbols) in the DNA string may be substituted for another symbol.

In order to correct the errors, current solutions require that multiple copies of a particular DNA string are generated. A majority rule is then applied to each index of the DNA string. For example, the most prominent symbol in each index of the DNA string is selected as the correct symbol. The symbols are converted to corresponding ones and zeros. The string of ones and zeros is then provided to decoder that implements a low-density parity-check (LDPC) code error correction scheme.

In traditional electronic storage systems, a big advantage of using LDPC codes is the ability to utilize soft bits (or soft information) to measure a reliability of a hard bit that was read from a memory cell. In DNA-based storage systems, the consensus data generated from the DNA string only includes hard bit information. As such, LDPC codes used by DNA-based storage systems cannot use soft bits or soft information to measure the reliability of the determined hard bit information. Accordingly, it would be beneficial for DNA-based storage systems to generate and use soft bit information to increase the efficiency and the reliability of its decoders that implement LDPC code error correction schemes.

### SUMMARY

The present application describes systems and methods for generating and updating soft bits, or soft information, for DNA-based storage systems. In an example, the soft information may be used to increase an efficiency of LDPC decoders of the DNA-based storage system. In an example, prior to or during a sequencing step, multiple copies of a particular DNA string or DNA sequence may be generated and/or read. Each DNA sequence is divided into one or more DNA segments having a segment length n (where n is equal to or greater than one). Corresponding DNA segments for each DNA sequence are compared to determine: 1) a ratio of DNA symbols in agreement in each of the DNA segments; and/or 2) whether a length of each of the DNA segments are the same (e.g., whether each DNA segment has the same number of DNA symbols). In some examples, the ratio of DNA symbols in agreement may be determined across the entire DNA segment or may be determined symbol by symbol (e.g., comparing all DNA symbols in a first position of each of the DNA segments then comparing all DNA symbols in the second position of each of the DNA segments, etc.).

The ratio and/or the length information may be used to determine a bit error rate (BER) of a particular DNA segment. The BER of each DNA segment may then be used to determine the magnitude levels of soft information, or a log likelihood ratio (LLR) of that particular DNA segment. The soft information may be used to increase the accuracy and efficiencies of LDPC codes used by a decoder of the DNA-based storage system.

In some examples, the magnitude levels of soft information may be determined in an “offline” environment (e.g., in a laboratory). In another example, the magnitude levels of soft information may be determined in real-time or substantially real-time in an “online” environment (e.g., when the DNA-based storage system is being used in the field). In addition, the systems and methods described also enable the magnitude levels of soft information to be updated in real-time or substantially real-time thereby further increasing the accuracy and efficiency of the LDPC codes used by the decoder of the DNA-based storage system.

Accordingly, the present application describes a method for generating soft information for a DNA-based storage system. In an example, to generate soft information, a first copy of a DNA sequence and a second copy of the DNA sequence are received from a DNA storage medium of the DNA-based storage system. The first copy of the DNA sequence is divided into a first DNA segment and a second DNA segment. Each of the first DNA segment and the second DNA segment of the first copy of the DNA sequence include at least one DNA symbol. The second copy of the DNA sequence is also divided into a first DNA segment and a second DNA segment. In an example, each of the first DNA segment and the second DNA segment of the second copy of the DNA sequence include at least one DNA symbol. The first DNA segment of the first copy of the DNA sequence is compared to the first DNA segment of the second copy of the DNA sequence to determine first consensus information and the second DNA segment of the first copy of the DNA sequence is compared to the second DNA segment of the second copy of the DNA sequence to determine second consensus information. A first bit error rate (BER) associated with the first DNA segment of the first copy of the DNA sequence is determined based, at least in part, on the first consensus information. Likewise, a second bit error rate (BER) associated with the second DNA segment of the first copy of the DNA sequence is determined based, at least in part, on the second consensus information.

The present application also describes a DNA-based storage system that includes a control system. The control system is operable to divide a plurality of copies of a DNA sequence into multiple DNA segments. The control system also compares corresponding DNA segments in each of the plurality of copies of the DNA sequences to determine at least one of a ratio of DNA symbols in agreement between corresponding DNA segments of the multiple DNA segments and a length consensus between corresponding DNA segments of the multiple DNA segments. The control system may generate soft information associated with each of the multiple DNA segments based, at least in part, on the at least one of the ratio of DNA symbols in agreement and the length consensus.

Also described is a DNA-based storage system that includes a dense storage system that stores a DNA sequence and at least one copy of the DNA sequence. The DNA-based storage system also includes a control system operably coupled to the dense storage system. The control system includes means for accessing the dense storage system to retrieve the DNA sequence and the at least one copy of the DNA sequence. The control system also includes means for dividing the DNA sequence and the at least one copy of the DNA sequence into corresponding DNA segments. In an example, each of the DNA segments include at least one DNA symbol. The control system also includes means for comparing the corresponding DNA segments to determine at least one of a ratio of the DNA symbols in agreement between the corresponding DNA segments and a length consensus between the corresponding DNA segments. The control system also includes means for generating soft information associated with each of the DNA segments based, at least in part, on the at least one of the ratio of the DNA symbols in agreement and the length consensus.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS

Non-limiting and non-exhaustive examples are described with reference to the following Figures.

FIG. 1 illustrates a DNA-based storage system according to an example.

FIG. 2 illustrates how a DNA sequence is divided into a number of DNA segments and how corresponding DNA segments of various copies of the DNA sequence are compared to determine soft bit information according to an example.

FIG. 3 illustrates an example method for generating soft bit information for a DNA-based storage systems according to an example.

FIG. 4A illustrates an initial log likelihood ratio (LLR) table according to an example.

FIG. 4B illustrates an updated LLR table in which the reliability metrics of the initial LLR table of FIG. 4A have been updated according to an example.

FIG. 5 illustrates a block diagram of a system that may be associated with or otherwise utilize various subsystems of the DNA-based storage system of FIG. 1 according to an example.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Examples may be practiced as methods, systems or devices. Accordingly, examples may take the form of a hardware implementation, an entirely software implementation, or an implementation combining software and hardware aspects. The following detailed description is therefore not to be taken in a limiting sense, and the scope of the present disclosure is defined by the appended claims and their equivalents.

As indicated above, DNA-based storage systems are emerging as a promising storage technology. However, DNA-based storage systems are more prone to errors when compared to traditional electronic data storage systems. For example, during synthesis (e.g., writing), storage and/or sequencing (e.g., reading), one or more symbols may be inserted into and/or removed from a DNA sequence (also referred to herein as a DNA string). In other examples, during synthesis, storage and/or sequencing, one or more symbols in the DNA sequence may be substituted for another symbol.

Some of these errors are typically corrected using a majority rule approach in which multiple copies of a particular DNA sequence are compared to each other and the most prominent symbol in each index of the DNA sequence is selected as the correct symbol. The symbols are converted to corresponding ones and zeros and the string of ones and zeros is provided to an LDPC decoder.

In traditional LDPC decoding, a decoder receives hard bit data from a memory die in response to an issued read command. After receiving the hard bit data, the decoder attempts to decode the data using only the hard bit data. If decoding with the hard bit is unsuccessful or fails, soft bit data may be used. Soft-bit data is used to achieve relatively high error correction rate. For example, the decoder operates on “soft” data, such as log-likelihood ratio (LLR) inputs, which indicate a data value and a probability that the data value is correct. However, the majority rule approach described above only generates hard bit data or “hard” data.

In order to address the above, the present application describes a DNA-based storage system that generates and updates soft bits, or soft information. The soft bits may be used to increase an efficiency of LDPC codes utilized by a decoder of the DNA-based storage system. In order to generate soft bits, the DNA-based storage system generates multiple copies of a particular DNA string or DNA sequence. Each DNA sequence is divided into one or more DNA segments having a segment length n (where n is equal to or greater than one). Corresponding DNA segments for each copy of the DNA sequence are compared to determine a ratio of DNA symbols in agreement in each of the DNA segments and/or if a length of each of the DNA segments are the same. In some examples, the ratio of DNA symbols in agreement may be determined across the entire DNA segment. In another example, the ratio of DNA symbols in agreement may be determined symbol by symbol or index by index across each of the DNA segments. Using this information, soft information (or soft bits) are generated.

For example, the ratio and/or the length information may be used to determine a consensus codeword associated with a particular DNA segment and an associated bit error rate (BER) of the consensus codeword. The BER of each consensus codeword (or DNA segment) may then be used to determine the magnitude levels of associated soft information also known as a log likelihood ratio (LLR) associated with the consensus codeword. The soft information may be used to increase the accuracy and efficiencies of LDPC codes used by the decoder of the DNA-based storage system.

In some examples, the magnitude levels of soft information may be determined “offline” environment (e.g., in a laboratory). In another example, the magnitude levels of soft information may be determined in real-time or substantially real-time in an “online” environment (e.g., when the DNA-based storage system is being used). In addition, the systems and methods described also enable the magnitude levels of soft information to be updated in real-time or substantially real-time thereby further increasing the accuracy and efficiency of the LDPC codes used by the decoder.

Accordingly, the present application includes many technical benefits including improving the correction capabilities of DNA-based storage systems by increasing the efficiency and accuracy of DNA-based storage system decoders. As the correction capabilities of DNA-based storage systems increases, DNA-based storage systems will be able to store longer DNA strings which in turn, enables greater storage density.

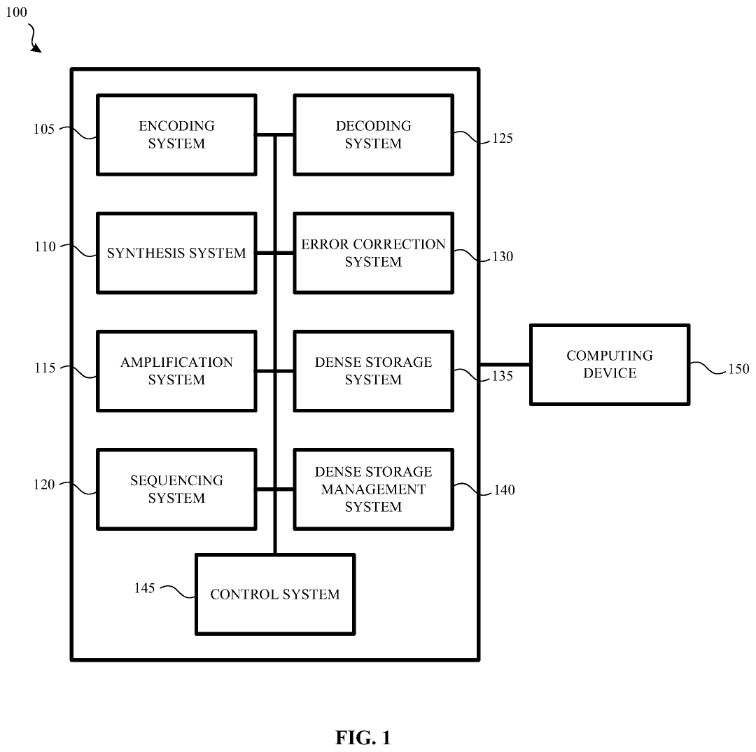

These and other examples will be described in more detail below with respect to FIG. 1-FIG. 5.

FIG. 1 illustrates a data storage system 100 according to an example. The data storage system 100 may be used to store data that is “more dense” when compared to data that is stored in a traditional electronic storage medium such as, for example, hard disks, optical disks, flash memory, and the like. For example, the data storage system 100 may be used to store synthetic DNA-based data.

DNA includes four naturally occurring nucleotide bases: adenine (A), cytosine (C), thymine (T) and guanine (G). In order to store data in synthetic DNA, received data is encoded to the various nucleotide bases. For example, data received as ones and zeros is encoded or otherwise mapped to various sequences of the synthetic DNA nucleotide bases. Once encoded, the data may be synthesized (e.g., written) and stored (e.g., in a dense storage system). To retrieve the stored data, the synthetic DNA molecules are sequenced (read) and subsequently decoded. As part of the decoding process, the synthetic DNA nucleotide bases are remapped to the original ones and zeros. Each of these processes will be discussed in greater detail below.

Although synthetic DNA-based data and associated DNA-based storage systems are specifically mentioned, the systems and methods described herein may be applicable to traditional electronic storage mediums/systems and/or traditional digital/binary data.

In an example, the data storage system 100 includes an encoding system 105. The encoding system 105 receives digital/binary information and/or data (e.g., ones and zeros) from a computing device (e.g., computing device 150) or from another source. When the data is received, the encoding system 105 converts or maps the ones and zeros of the original data into various DNA sequences using the synthetic DNA nucleotide bases ACTG. For example, the DNA nucleotide base “A” may be assigned a value 00, the DNA nucleotide base “C” may be assigned a value 01, the DNA nucleotide base “T” may be assigned a value 10 and the DNA nucleotide base “G” may be assigned a value 11.

Using this encoding scheme, digital data of 010010110100 would be represented as a DNA sequence or DNA string of CATGCA. Although specific values for each of the DNA nucleotide bases are given, each nucleotide base may be assigned any value. Additionally, although a specific encoding scheme is discussed, any encoding scheme may be used by the encoding system 105.

The data storage system 100 may also include a synthesis system 110. In an example, the synthesis system 110 writes or otherwise manufactures DNA strands based on the data provided by the encoding system 105. For example, using a series of chemical steps or processes, the various DNA bases (e.g., the ACTG bases determined in the encoding process) are assembled to mirror the encoded data. Although chemical steps or processes are mentioned, the synthesis system 110 may use other synthesis techniques.

Continuing with the example above, since the digital data of 010010110100 is represented as CATGCA, the synthesis system 110 would first generate and/or identify a “C” base. An “A” base would then be generated and/or identified and be attached to the “C” base. A “T” base would then be generated and/or identified and be attached to the “CA” combination that was previously generated. This process repeats until the entire DNA sequence (e.g., CATGCA) is generated.

When the synthesis process is complete, the DNA sequence is stored in a physical storage medium such as, for example, a dense storage system 135. The dense storage system 135 enables the synthesized DNA sequence to be stored and subsequently accessed. In an example, any storage medium capable of storing DNA-based data may be used as the dense storage system 135.

Once the DNA sequence has been stored, it may be subsequently accessed and prepared for sequencing (e.g., being read). As part of the preparation process, multiple copies of the DNA sequence may be generated. In an example, an amplification system 115 of the data storage system 100 may ensure that multiple copies of the DNA data are generated.

A sequencing system 120 may then be used to read DNA sequences from the dense storage system 135. In an example, the sequencing system 120 determines and/or identifies an order of the DNA symbols (e.g., ACTG) in a DNA segment of a DNA sequence that is being read. The sequencing system 120 may use a variety of sequencing methods such as, for example, sequencing by synthesis, nanopore sequencing, and the like.

Once the DNA sequence has been read, a decoding system 125 maps the DNA symbols back to digital data. For example, if the decoding system 125 receives CATGCA as the DNA sequence, the decoding process performed by the decoding system 125 would return 010010110100 to a requesting computing device (e.g., computing device 150).

In some examples, errors may occur during the synthesis process, the storage process and/or the sequencing process. These errors may be insertion and deletion (indel) errors and/or substitution errors. For example, during a synthesis process in which the DNA sequence CATGCA is being synthesized, one or more symbols may be deleted or lost. As a result, a DNA sequence CTGCA may be stored by the dense storage system 135. In another example, during a synthesis process in which the DNA sequence CATGCA is being synthesized, an additional symbol may be added. As a result, a DNA sequence CCATGCA may be stored by the dense storage system 135. Although a single insertion error and a single deletion error are discussed, multiple deletions and/or insertions may occur in a synthesis process. Additionally, these errors may occur during storage and/or during a sequencing process.

In yet another example, during a synthesis process in which the DNA sequence CATGCA is being synthesized, the synthesis system 110 may substitute one symbol for another. As a result, a DNA sequence TATGCA may be stored in the dense storage system 135 instead of the DNA sequence CATGCA. In an example, multiple substitution errors (along with one or more indel errors) may occur during the synthesis process, during storage and/or during a sequencing process.

In order to address the above, the data storage system 100 may include an error correction system 130. The error correction system 130 may be part of the decoding system 125. The error correction system 130 may use various processes to detect and address indel errors and/or substitution errors. In one example, indel errors may be addressed by generating multiple copies of a particular DNA sequence. Once generated, the copies of the DNA sequence are read and compared to generate a consensus codeword. For example, a first DNA symbol (or DNA segment consisting of multiple DNA symbols) of a first DNA sequence is compared with a first DNA symbol (or DNA segment consisting of multiple DNA symbols) from one or more of the copies of the DNA sequence. This process repeats for each DNA symbol (or DNA segment) in the DNA sequence.

The error correction system 130 may then determine, based on consensus data across all of the copies, which DNA symbol is the correct (or most correct) DNA symbol for that particular index (or DNA segment). The most prominent DNA symbol in each index of the DNA sequence is selected as the correct DNA symbol and a consensus codeword is generated for each DNA segment. The resulting consensus codeword is mapped to corresponding ones and zeros and is provided to a LDPC decoder.

In an example, the consensus data generated by the error correction system 130 may be referred to herein as hard bit data or hard information. The error correction system 130 and/or the decoding system 125 described in the present application may also generate and use soft-bit data or soft information using information associated with the consensus data (or using information that is obtained while the consensus data is determined).

For example, once multiple copies of a particular DNA sequence has been copied or otherwise generated (e.g., by the amplification system 115), each copy of the DNA sequence that is associated with a received codeword (e.g., DNA-based data that is to be read from the dense storage system 135), is divided into k DNA segments (where k is equal to or greater than two). Each DNA segment has a DNA segment length n (where n is equal to or greater than one).

For example, the DNA sequence CATGCA may be divided into two different DNA segments having a length of three. As such, a first DNA segment may be “CAT” and a second DNA segment may be “GCA”. In another example, the DNA sequence CATGCA may be divided into three different DNA segments having a length of two. In this example, the first DNA segment would be “CA”, the second DNA segment would be “TG” and the third DNA segment would be “CA”. In yet another example, the DNA sequence CATGCA may be divided into six different DNA segments having a length of one. In this example, the first DNA segment would be “C”, the second DNA segment would be “A”, the third DNA segment would be “T”, the fourth DNA segment would be “G” and so on. The position of each DNA symbol in each DNA segment is referred to as an index. Thus, in the DNA segment “CAT”, “C” is the first index, “A” is the second index and “T” is the third index.

Corresponding DNA segments for each copy of a DNA sequence associated with a codeword (or data to be read) are compared to determine: 1) a ratio of DNA symbols in agreement in each index of each of the DNA segments; and/or 2) whether a length of each of the DNA segments are the same. Using this information, soft bit information (or the log likelihood ratio (LLR)) of that particular consensus codeword (e.g., a DNA segment, a DNA symbol, a series of bits, or a bit), may be generated. In some examples, a single LLR value may be generated for an entire consensus codeword. In another example, each bit(s) or DNA symbol in a particular DNA segment may be analyzed separately to determine an LLR for that DNA symbol or bit.

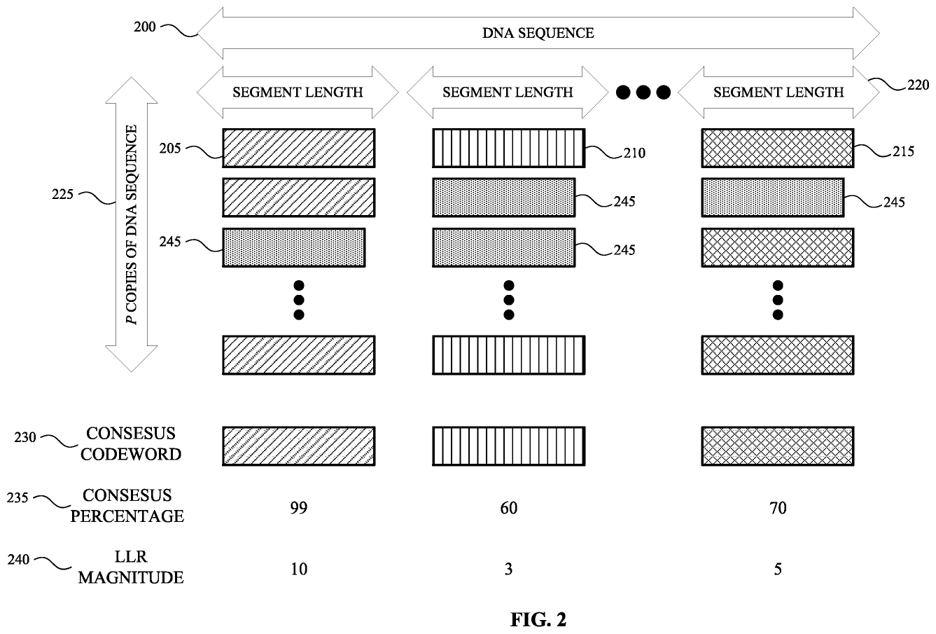

In the example shown in FIG. 2, a DNA sequence 200 is divided into k different DNA segments with each DNA segment having a DNA segment length n 220. For example, the DNA sequence 200 may be divided into a first DNA segment 205, a second DNA segment 210 and a k DNA segment 215. Although a total of k DNA segments are shown, the DNA sequence 200 may be divided into any number of DNA segments.

In FIG. 2, the first DNA segment 205 is represented by a first shaded box and has an associated length, the second DNA segment 210 is represented by a second shaded box and has an associated length and the k DNA segment 215 is represented by a third shaded box and has an associated length. DNA segments that are not in consensus with corresponding DNA segments in a particular grouping are represented by the shaded box 245. A DNA segment is not in consensus with other DNA segments in a particular grouping if the length of the DNA segment is different than the length of the other DNA segments in the grouping (e.g., represented by the shorter length of the shaded box 245) and/or one or more DNA symbols in the DNA segment do not match one or more DNA symbols in the other DNA segments in the grouping.

As discussed above, multiple copies (represented as p, in which p is equal to or greater than 1) of the DNA sequence 225 may be generated (e.g., using the amplification system 115) and/or read in response to a decoding system (e.g., decoding system 125) receiving a codeword or otherwise receiving a read request. Corresponding DNA segments for each copy of the DNA sequence 225 to be read are compared to determine a consensus codeword 230. Depending on the ratio of symbols in agreement for each copy of the DNA sequences 225 and/or on whether a length of each of the DNA segments are the same (referred to as consensus information), a bit error rate (BER) is determined for the consensus codeword 230. In another example, the BER may be determined for each DNA symbol or DNA segment.

In the examples that follow, a BER is specifically mentioned for binary LDPC decoding. However, a symbol error rate (SER) may be used for non-binary LDPC decoding. In binary decoding, a LLR for a given bit of a codeword may be defined as the log of the probability of the given bit being a logic 0 value divided by the probability of the given bit being a logic 1 value. In non-binary decoding, a LLR for a given segment or symbol may be defined as the log of the probability of the given symbol being one of the four symbols (e.g., ACGT) divided by the probability of the given symbol being another one of the four symbols. It should be appreciated that although the following examples and description are described with respect to binary LDPC decoding, the same or similar principles can be applied to implementations in which non-binary LDPC decoding is used.

Once the BER for each consensus codeword 230 (e.g., a DNA symbol, a DNA segment and/or associated bits or a bit) is determined, a LLR for the consensus codeword 230 is determined. As indicated above, the LLR of the consensus codeword 230 may be equivalent to soft bit(s) value or soft information. As such, in order to determine the LLR magnitude of the consensus codeword 230, the following equation may be used:

$$\vert {LLR}_{r} \vert = log \frac{1 - {BER}_{r}}{{BER}_{r}}$$

In the equation above, BERᵣ is the bit error rate of a particular reliability bin r. In an example, each reliability bin r may correspond to a particular consensus codeword 230 associated with DNA segments or DNA symbols and each reliability bin r may have an associated consensus percentage or reliability.

For example and referring back to FIG. 2, the consensus percentage 235 for the first DNA segment 205 and/or the consensus codeword 230 associated with the first DNA segment 205, is 99%. The consensus percentage 235 for the second DNA segment 210 and/or the consensus codeword 230 associated with the second DNA segment 210, is 60% and the consensus percentage 235 for the k DNA segment 220 and/or the consensus codeword 230 associated with the k DNA segment 215, is 70%. As indicated above, the range of agreement for each of the consensus codewords and/or DNA segments may be used to determine or may otherwise be associated with each of the r reliability bins. An LLR magnitude 240 may then be determined for each reliability bin r.

For example, the decoding system 125 (FIG. 1) may determine that because 99% of the copies of the first DNA segment 205 agree on its DNA symbol value(s) and/or length, a LLR or LLR magnitude 240 of “10” may be associated with this reliability bin. The decoding system may also determine that because 60% of the copies of the second DNA segment 210 agree on its symbol value(s) and/or length, a LLR or LLR magnitude 240 of “3” may be associated with this reliability bin. Likewise, the decoding system may determine that because 70% of the copies of the k DNA segment 215 agree on its symbol value(s) and/or length, a LLR or LLR magnitude 240 of “5” may be associated with this reliability bin.

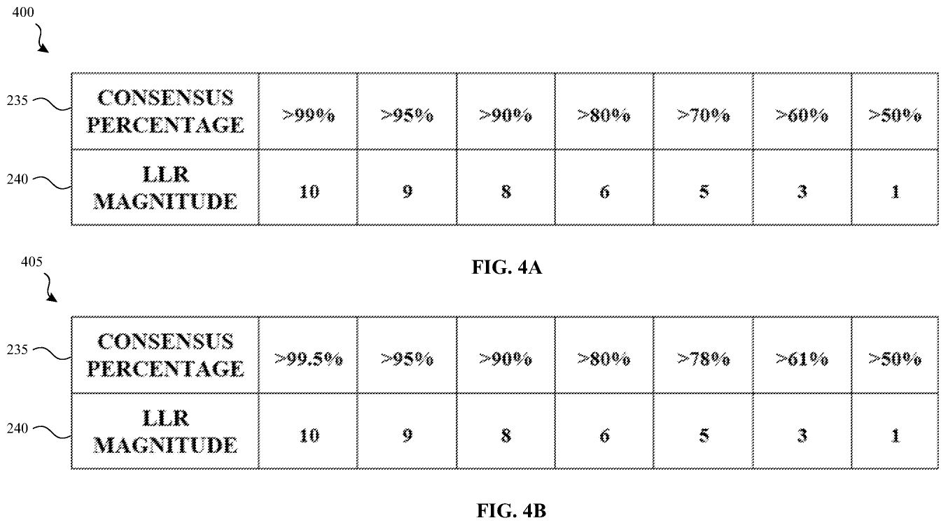

FIG. 4A illustrates an initial log likelihood ratio (LLR) table 400 according to an example. For example and as shown with respect to FIG. 4A, the consensus percentages 235 and associated LLR magnitudes 240 may be provided or otherwise stored in the LLR table 400. Although the LLR table 400 shows seven different reliability bins or sections, with each section having specific consensus percentages 235 and LLR magnitudes 240, these are for example purposes only and it is contemplated that any number of reliability bins may be generated and associated with any number of consensus percentages 235 and LLR magnitudes 240.

In some examples, some of the initial values for the LLR table 400 may be generated “offline”. In other examples, some of the initial values for the LLR table 400 may be generated in the field in real time or substantially real-time. In either case, the consensus percentages 235 provided in the LLR table 400 and/or the LLR magnitude 240 values may be adjusted in real-time or substantially real-time such as will be described in greater detail below.

Referring back to FIG. 1, the data storage system 100 may also include a dense storage management system 140. In an example, the dense storage management system 140 controls the various operations and/or processes that are carried out by and/or on the dense storage system 135. The operations and/or processes may include the mechanics of storage and retrieval of the DNA data and/or information storage management (e.g., making copies of data, deleting data, selecting subsets of the data, etc.).

The data storage system 100 may also include a control system 145. The control system 145 may include at least one processor, at least one controller and/or other such control circuitry. The control system 145 may include circuitry for executing instructions from the computing device 150 (or from another source) and/or providing instructions to the various subsystems of the data storage system 100.

In an example, the data storage system 100 may be associated with or otherwise communicatively coupled to a computing device 150. The computing device 150 may, via a communication channel, provide data and/or instructions to the data storage system 100. The computing device 150 may also receive data from the data storage device 100 via the communication channel. Although FIG. 1 shows the data storage system 100 being separate from the computing device 150, the data storage system 100, and/or one or more subsystems of the data storage system 100, may be integrated with the computing device 150.

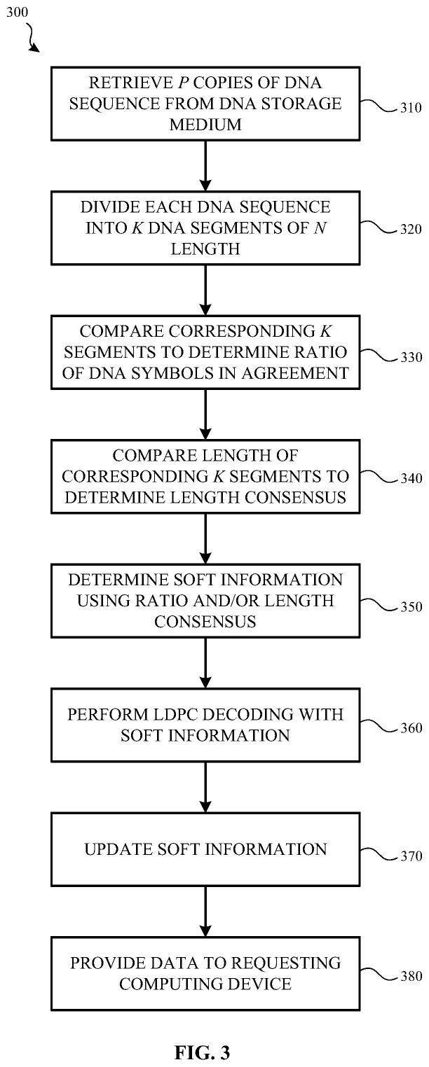

FIG. 3 illustrates an example method 300 for generating soft bit information for a DNA-based storage systems according to an example. In an example, one or more of the operations shown and described with respect to FIG. 3 may be performed by one or more subsystems of the DNA-based storage system 100 shown and described with respect to FIG. 1.

Method 300 begins when multiple (e.g., p) copies of a particular DNA sequence are retrieved (310) and/or read from a DNA storage medium. In some example, the particular DNA sequence is associated read request or other such request to retrieve DNA-based data received by the DNA-based storage system. In an example, the particular DNA sequence may be associated with a particular codeword or codewords that are identified in or are otherwise associated with the request.

When the p copies of the particular DNA sequence have been retrieved, the DNA sequence and/or each copy of the DNA sequence is divided (320) into k DNA segments, with each DNA segment having n length. In an example, k is equal to or greater than two and n is equal to or greater than one. For example, the DNA sequence may be divided into a first DNA segment and a second DNA segment. Further, the first DNA segment and the second DNA segment may each have three DNA symbols (e.g., have a length of three).

Each k DNA segment of each copy of the DNA sequence is compared (330) to corresponding DNA segments to determine a ratio of DNA symbols in agreement. For example, a first DNA segment in the particular DNA sequence is compared to a corresponding first DNA segment in a first copy of the particular DNA sequence and/or a first DNA segment in all the other copies of the particular DNA sequence. Likewise, a second DNA segment in the particular DNA sequence is compared to a corresponding second DNA segment in all the other copies.

In addition, a length of each of the k DNA segments of each copy of the DNA sequence is compared (340) to the lengths of corresponding p DNA segments to determine whether each DNA segment has the same number of DNA symbols. For example, a length of the first DNA segment in the particular DNA sequence is compared to a length of the corresponding first DNA segment in the first copy of the particular DNA sequence and/or to a length of the first DNA segment in all other copies of the particular DNA sequence. Likewise, a length of the second DNA segment in the particular DNA sequence is compared to a length of the corresponding second DNA segment in all other copies.

Although FIG. 3 illustrates operation 330 and operation 340 occurring sequentially, each of operations 330 and 340 may occur simultaneously or substantially simultaneously. Additionally, it is contemplated that operation 330 may be performed without performing operation 340. Likewise, it is contemplated that operation 340 may be performed without performing operation 330. For example, the BER of a particular DNA segment may be based on the ratio information alone, the length consensus information alone, or a combination of the ratio information and the length consensus.

Once the agreement ratio of the DNA segments and/or the length consensus information of the DNA segments have been determined, soft information may be determined (350) using the agreement ratio and/or the length consensus information. As discussed above, the agreement ratio and/or the length consensus information may closely resemble or otherwise be equivalent to a BER of the particular DNA segment or consensus codeword. Using the BER, one or more reliability bins and associated LLR values for the reliability bins may be generated such as previously described.

When the LLR values have been generated, LDPC decoding may be performed (360) using the soft information. In an example, the LDPC decoding may be binary LDCP decoding or non-binary LDPC decoding.

During the decoding process, the LLR values may be updated (370). Updating LLR values will be described in more detail below. In some examples, although operation 370 is shown as occurring sequentially after operation 360, operation 360 and operation 370 may occur simultaneously or substantially simultaneously. In some examples, updating the soft information may begin after a threshold number of decoding operations have been performed. Upon completion of the LDPC decoding, the decoded data is provided (380) to a requesting computing device.

In some examples and as indicated above, the soft information or LLR values may be generated offline. However, these offline values may be updated in real-time or substantially real-time during a decoding process. The following description describes a process for updating LLR values during a binary LDPC decoding process. However, the same or similar operations may be performed during a non-binary LDPC decoding process. Updating the soft information or the LLR values may be completed by one or more subsystems of the data storage device 100 shown and described with respect to FIG. 1.

As described above, the consensus information for the various DNA segments may be used to generate reliability metrics or LLR values (also referred to as an LLR magnitude). However, these LLR values may be updated in real-time or substantially real-time. As an example and as will be as described in further detail below, the LLR values that are initially generated (whether offline or online) include a priori LLR values and a posteriori LLR values. From the examples described above, the a priori LLR values may be those that are shown and described with respect to FIG. 4A while the a posteriori LLR values may be those that are shown and described with respect to FIG. 4B. For example, FIG. 4B illustrates an updated LLR table 405 in which the reliability metrics of the initial LLR table 400 of FIG. 4A have been updated according to an example. For example, in FIG. 4B, the soft bit information update process described below may cause the consensus percentage 235 in the first reliability bin to change from 99% to 99.5%, cause the consensus percentage 235 in the fifth reliability bin to change from 70% to 78% and cause the consensus percentage 235 in the sixth reliability bin to change from 60% to 61%.

In another example, the a priori LLR values may be initial magnitude levels or values associated with the soft information and the a posteriori LLR values may be magnitude levels or values that are modified or are otherwise determined during at least part of a decoding process. For example, the magnitude levels or values may be associated with the LLR magnitude 240. As such, one or more of the LLR magnitudes 240 may be updated based on the processes described herein.

The magnitude levels or values may indicate a likelihood, reliability, or confidence level of a particular decoded value being correct. Thus, the greater the magnitude level or value, the higher the reliability and/or the higher the likelihood that the decoded value is correct. Conversely, the lower the magnitude level or value, the lower the reliability and/or the lower the likelihood that the decoded value is correct.

In an example, a priori LLR values P are an initial set of LLR values for the DNA segments described above. In essence, the a priori LLR values are initial LLR value estimates. Over time and/or during a LDPC decoding process, it may be determined that the a priori LLR values P should be updated to increase the accuracy and/or the efficiency of a decoder system of a DNA-based storage device. As such, one or more of the a priori LLR values (e.g., a sign component and/or a magnitude component) may need to be updated.

LLR values that are updated (e.g., during the LDPC decoding process) are referred to as a posteriori LLR values Q. Any changes to the a priori LLR values P may reflected or indicated in the set of a posteriori LLR values Q.

As discussed above, initial a priori LLR values may be determined using DNA segment ratio information and/or DNA segment length consensus. These initial LLR values are referred to as a priori LLR values P^{init} for each consensus codeword (e.g., consensus codeword 230 (FIG. 2) of a DNA segment. As described in further detail below, the initial a priori LLR values P^{init} may be updated to form a new set of a priori LLR values P^{new} at least once during a DNA sequence decoding operation.

In some examples, each initial a priori LLR value may correspond to a particular reliability bin such as described above. For example, each reliability bin may have an associated a priori LLR magnitude value. Thus, in an example configuration that utilizes three reliability bins, a first reliability bin may be associated with a first a priori LLR magnitude value, a second reliability bin may be associated with a second a priori LLR magnitude value, and a third reliability bin may be associated with a third a priori LLR magnitude value. For a given DNA segment or consensus codeword (e.g., consensus codeword 230 (FIG. 2)), the decoding system 125 and/or the error correction system 130 may be configured to select which of the plurality of a priori magnitude values to assign to a particular codeword based on a determined reliability bin. As described above, the a priori LLR magnitude values may be included in an a priori LLR table (e.g., LLR table 400 (FIG. 4A)).

In an example, during a DNA sequence decoding process, the decoding system may access the a priori LLR table. If the DNA sequence is decoded successfully during the initial decoding process, using the soft information from the LLR table, the control system 145 of the data storage system 100 may determine that the LLR values do not need to be updated. However, if the DNA sequence is not properly decoded, the decoding system 125 and/or the control system 145 may determine that the LLR values in the LLR table should be updated. Any updated LLR P^{init} values will be referred to as a set of a posteriori LLR values Q (such as will be described in more detail below).

The initial a priori LLR magnitude values may correspond to an underlying memory error model. The memory error model utilizes a reliability characteristic for identifying the a priori magnitude values. As discussed above, the reliability characteristic is a bit error rate (BER) for binary decoding and a symbol error rate (SER) for non-binary decoding.

Because the BERs of each of the DNA segments may be generated offline, the underlying memory error model may not provide optimal estimations of the LLR values (e.g., the initial a priori magnitude values). As a result, the DNA sequence decoding process may not be as efficient as it could be.

As such, instead of just calculating an initial set of a priori LLR values P^{init} and then performing LDPC decoding using these values, a positive feedback process is implemented in which after a portion of the decoding process is performed and before the decoding is completed, information obtained as a result of performing the portion, as reflected in a current set of a posteriori LLR values Q^{cur} generated during the portion, is used to update or improve the reliability characteristic of the memory error model. This information is then used to generate a new/updated set of a priori LLR values P^{new} and a new/updated set of a posteriori LLR values Q^{new}. The new/updated set of posteriori LLR values Q^{new} is then used for a subsequent portion of the decoding process.

Calculating the new/updated a posteriori LLR values Q^{new} and using those values during subsequent portions of the decoding process may enable the DNA decoding process to be completed faster when compared to decoding using the initial LLR values. The reliability characteristic of the memory error model, and in turn new/updated sets of a priori and a posteriori LLR values P^{new}. Q^{new} may be updated once or alternatively multiple times during the entire process.

In an example, the control system 145, either alone or in conjunction with the decoding system 125 and/or the error correction system 130 (or other systems of the data storage system 100), may be configured to control the starting, stopping or pausing of the DNA sequence decoding process. In one example, the decoding system 125 may stop or pause the decoding process when a number of iterations has reached or exceeded a threshold level. In an example, an iteration may be defined by one cycle through the variable nodes for a certain aspect of the decoding process. As another example, the decoding system 125 may stop or pause the decoding process upon determining that the decoding process is stuck or not progressing at a fast enough rate.

When the decoding system 125 causes the decoding process to stop, the a posteriori LLR values Q in their current state may be referred to as the current a posteriori LLR values Q^{cur}. During this time, new/updated a priori LLR values P^{new} and a posteriori LLR values Q^{new} may be calculated using the process described below.

In a first operation, the decoding system 125 may access the current a posteriori LLR values Q^{cur} and calculate updated reliability characteristic values for each of the bits (or symbols) of the codeword based on the current a posteriori LLR values Q^{cur}. In an example, the reliability characteristic values are updated values in the sense that they are updated compared to the initial reliability characteristic values defining an initial memory error model upon which the initial a priori LLR values are based. In other words, the updating of the reliability characteristic values is a refining of the initial memory error model for a given read codeword that may be different that the initially assumed memory error model.

As discussed above, the reliability characteristic of the memory model may be bit error rate for binary LDPC decoding or may be a symbol error rate for non-binary LDPC decoding. Assuming that log base 2 LLR values are used as the reliability metric, the following mathematical equation may be used:

$${BER}_{i} = \{ \begin{matrix}\frac{{2}^{- \vert {Q}_{i}^{cur} \vert}}{1 + {2}^{- \vert {Q}_{i}^{cur} \vert}} , & if sign ( {Q}_{i}^{cur} ) = {HB}_{i} \\ \frac{1}{1 + {2}^{- \vert {Q}_{i}^{cur} \vert}} , & if sign ( {Q}_{i}^{cur} ) \neq {HB}_{i}\end{matrix}$$

In this equation, BERᵢ represents an ith bit error rate estimation for an ith bit of the codeword, |Qᵢ^{cur}|represents the magnitude component of the ith current a posteriori LLR Qfᵢ^{cur}. HBᵢ represents the hard bit value of the associated hard bit/soft bit combination value of the ith bit, which may also be equal to and/or correspond to a sign component of the associated ith initial a priori LLR Pᵢ^{init}. Although this equation leverages the log base 2 relationship between bit error rate and log likelihood ratios, a log base may also be used such as described above. Further, the ith bit may be a single bit representation of a DNA symbol in a DNA segment.

Upon calculating estimated bit error rates for each of the bits, the estimated bit error rates may then be used to change an LLR value of a particular reliability bin. For example, estimated bit error rates may be used to calculate average or expected, estimated bit error rates BERᵣ=E_{i∈t}[BERᵢ] for each reliability bin. For example, each of the ith BERᵢ values associated with each of the ith bits may be grouped into a respective reliability bin. Using the reliability bin information, the reliability bin that each ith BERᵢ value is associated with may be determined. Upon grouping each of the ith BERᵢ values into their respective reliability bins, an estimated bit error rates BERᵣ for each of the reliability bins may be determined.

In one example, the average, estimated bit error rates for each of the reliability bins may be used to calculate new/updated a priori LLR values according to the following mathematical equation:

$${P}_{i ∈ r}^{new} = ( 1 - 2 \cdot {HB}_{i} ) \cdot {log}_{2} \frac{1 - {BER}_{r}}{{BER}_{r}}$$

In the above equation, Pᵢ^{new} represents a new or updated a priori LLR for an ith bit, HBᵢ is the hard bit logic value of the ith bit, and BERᵣ is the average, estimated BER of the rth reliability bin. The term “i∈r” is used to denote that the average, estimated BER that is used is the one associated with the reliability bin with which the ith bit is also associated.

For example, suppose the first bit of the codeword is grouped into a first reliability bin. In turn, the average, estimated BERᵣ value used when calculating a new a priori LLR Pₗ^{new} for the first bit would be the first average, estimated BER determined for the first reliability bin.

Upon calculating the new a priori LLR values P^{new} for the bits of the codeword, the new a priori LLR values P^{new} is used calculate new/updated a posteriori LLR values Q^{new} according to the following mathematical equation:

Qᵢ^{new}=Qᵢ^{cur}−Pᵢ^{old}+Pᵢ^{new}

In the above. Qᵢ^{new} represents the new/updated a posteriori LLR for the ith bit, Qᵢ^{cur} represents the current a posteriori LLR for the ith bit when the decoding process is stopped or paused, Pᵢ^{new} represents the new/updated a priori LLR for the ith bit, and Pᵢ^{old} represents the old a priori LLR for the ith bit.

For an initial update process (i.e., an update process performed after the decoding process is stopped or paused for the first time), the old a priori LLR values P^{old} may be set to the initial a priori LLR values P^{init}. Thereafter, for any subsequent update processes, the old a priori LLR values P^{old} may be set to the last “new” a priori LLR values determined in the prior update process.

When the new a posteriori LLR values Q^{new} have been determined, the decoding process may be resumed. In response, the decoding system 125 may access the new a posteriori LLR values Q^{new} and resume decoding using these values. This process may be repeated the DNA sequence is decoded. The above process is described in more detail in U.S. Pat. No. 10,554,227, entitled Decoding Optimization for Channel Mismatch, by Sharon et al., the entire disclosure of which is hereby incorporated by reference in its entirety.

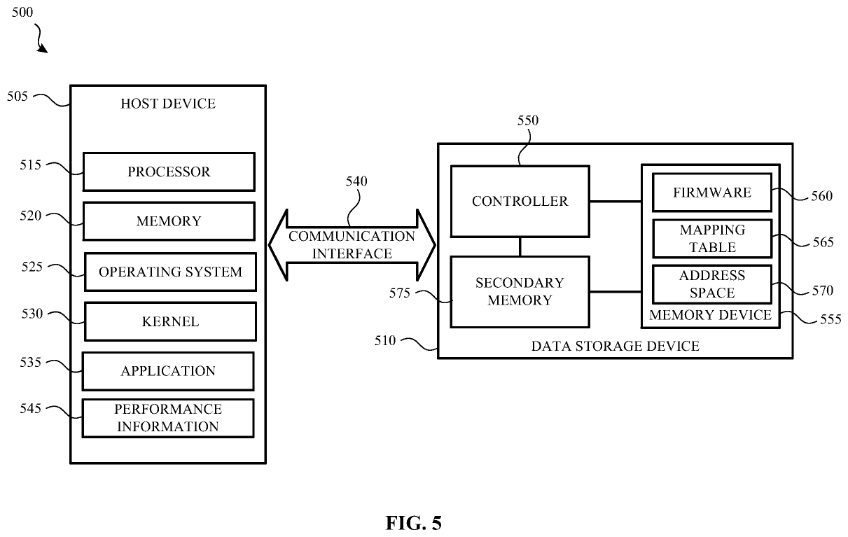

FIG. 5 is a block diagram of a system 500 that includes a host device 505 and a data storage device 510 according to an example. In an example, the host device 505 may be similar to the computing device 150 shown and described with respect to FIG. 1. The host device 505 includes a processor 515 and a memory device 520 (e.g., main memory). The memory device 520 may include an operating system 525, a kernel 530 and/or an application 535.

The processor 515 can execute various instructions, such as, for example, instructions from the operating system 525 and/or the application 535. The processor 515 may include circuitry such as a microcontroller, a Digital Signal Processor (DSP), an Application-Specific Integrated Circuit (ASIC), a Field Programmable Gate Array (FPGA), hard-wired logic, analog circuitry and/or various combinations thereof. In an example, the processor 515 may include a System on a Chip (SoC).

In an example, the memory device 520 can be used by the host device 505 to store data used by the processor 515. Data stored in the memory device 520 may include instructions provided by the data storage device 510 via a communication interface 540. The data stored in the memory device 520 may also include data used to execute instructions from the operating system 525 and/or one or more applications 535. In an example, the memory 520 is volatile memory, such as, for example, Dynamic Random Access Memory (DRAM).

In an example, the operating system 525 may create a virtual address space for the application 535 and/or other processes executed by the processor 515. The virtual address space may map to locations in the memory device 520. The operating system 525 may include or otherwise be associated with a kernel 530. The kernel 530 may include instructions for managing various resources of the host device 505 (e.g., memory allocation), handling read and write requests and so on.

The communication interface 540 communicatively couples the host device 505 and the data storage device 510. The communication interface 540 may be a Serial Advanced Technology Attachment (SATA), a PCI express (PCIe) bus, a Small Computer System Interface (SCSI), a Serial Attached SCSI (SAS), Ethernet, Fibre Channel, or WiFi. As such, the host device 505 and the data storage device 510 need not be physically co-located and may communicate over a network such as a Local Area Network (LAN) or a Wide Area Network (WAN), such as the internet. In addition, the host device 505 may interface with the data storage device 510 using a logical interface specification such as Non-Volatile Memory express (NVMe) or Advanced Host Controller Interface (AHCI).

The data storage device 510 includes a controller 550 and a memory device 555 (e.g. volatile and/or non-volatile memory). The memory device 555 (and/or portions of the memory device 555) may also be referred to as a storage medium. The memory device 555 includes a number of storage elements. In an example, each storage element is a chip or a memory die that is used to store data.

For example, the memory device 555 may include a first memory die and a second memory die. In an example, the first memory die and the second memory die include non-volatile memory elements such as, for example, NAND flash memory elements and/or NOR flash memory elements. Although two memory dies are mentioned, the memory device 555 may include any number of storage elements. For example, the storage elements may take the form of solid-state memory such as, for example, 2D NAND, 3D NAND memory, multi-level cell memory, triple level cell memory, quad-level cell memory, penta-level cell memory or any combination thereof.

The controller 550 may include circuitry for executing instructions. The instructions may originate from firmware 560 associated with the data storage device 510. In another example, the instructions may originate from the host device 505. Accordingly, the controller 550 may include circuitry such as one or more processors, a microcontroller, a DSP, an ASIC, an FPGA, hard-wired logic, analog circuitry and/or a combination thereof. In another example, the controller 550 may include a SoC.

The data storage device 510 may also include secondary memory 575. The secondary memory 575 may be a rotating magnetic disk or non-volatile solid-state memory, such as flash memory. While the description herein refers to solid-state memory generally, it is understood that solid-state memory may comprise one or more of various types of memory devices such as flash integrated circuits, NAND memory (e.g., single-level cell (SLC) memory, multi-level cell (MLC) memory (i.e., two or more levels), or any combination thereof), NOR memory, EEPROM, other discrete Non-Volatile Memory (NVM) chips, or any combination thereof.

In some examples, the memory device 555 is capable of storing data at a byte-addressable level, as opposed to other types of non-volatile memory that have a smallest writable data size such as a page size of 4 KB or a sector size of 512 Bytes.

In some examples, the memory device 555 may also store a mapping table 565 and/or an address space 570. In some examples, the controller 550 can associate portions of data stored in the secondary memory 575 with unique identifiers. The unique identifiers may be stored in the memory device 555 and be used by the operating system 525 to access stored data. For example, the mapping table 565 can provide a mapping of unique identifiers with indications of physical locations (e.g., Physical Block Addresses (PBAs)) where the corresponding portions of data are stored in the memory device 555 and/or the secondary memory 575.

In some examples, the firmware 560 may store, maintain, be associated with or otherwise have access to a mapping table (e.g., mapping table 565) that stores and/or maintains mapping information for the various DNA sequences such as described above.

As briefly discussed above, the memory device 555 may also include address space 570. The address space 570 can serve as at least a portion of an address space used by the processor 515. In an example, the address space 570 can store data at a byte-addressable level that can be accessed by the processor 515 (e.g., via the communication interface 540).

For example, the data storage device 510 may provide the host device 505 with an indication of the address space 570. The host device 505 may then associate an address range for the address space 570 and an indication that this address range is to be used as a byte-addressable address space, such as for a page cache.

In another example, the host device 505 may manage the data storage device 510 such that the processor 515 can directly access address space 570. For example, the data storage device 510 may provide logical to physical address translation information to the host device 505, which can be called by the host device 505 and executed by the processor 515 and/or the controller 550. In some examples, the controller 550 may include or otherwise be associated with a flash translation layer (FTL). The FTL may map the logical block addresses to the physical addresses of the memory device 555.

Although FIG. 5 illustrates the host device 505 being separate from the data storage device 510, the host device 505 and the data storage device 510, as well the various components described, may be part of a single device or part of multiple devices.

According to the examples described herein, aspects of the present application describe a method for generating soft information for a DNA-based storage system, comprising: receiving, from a DNA storage medium of the DNA-based storage system, a first copy of a DNA sequence and a second copy of the DNA sequence; dividing the first copy of the DNA sequence into a first DNA segment and a second DNA segment, each of the first DNA segment and the second DNA segment of the first copy of the DNA sequence including at least one DNA symbol; dividing the second copy of the DNA sequence into a first DNA segment and a second DNA segment, each of the first DNA segment and the second DNA segment of the second copy of the DNA sequence including at least one DNA symbol; comparing the first DNA segment of the first copy of the DNA sequence to the first DNA segment of the second copy of the DNA sequence to determine first consensus information; comparing the second DNA segment of the first copy of the DNA sequence to the second DNA segment of the second copy of the DNA sequence to determine second consensus information; determining, based at least in part, on the first consensus information, a first bit error rate (BER) associated with the first DNA segment of the first copy of the DNA sequence; and determining, based at least in part, on the second consensus information, a second bit error rate (BER) associated with the second DNA segment of the first copy of the DNA sequence. In an example, the first consensus information comprises a determination of a ratio of DNA symbols in agreement between the first DNA segment of the first copy of the DNA sequence and the first DNA segment of the second copy of the DNA sequence. In an example, the first consensus information comprises length information of the first DNA segment of the first copy of the DNA sequence compared to length information of the first DNA segment of the second copy of the DNA sequence. In an example, the method further comprises associating a reliability bin with a log likelihood ratio (LLR), the log likelihood ratio being associated with the first DNA segment of the first copy of the DNA sequence. In an example, the log likelihood ratio associated with the first DNA segment of the first copy of the DNA sequence is determined based, at least in part, on the first bit error rate. In an example, the log likelihood ratio is determined in an offline environment. In an example, the method also includes updating the log likelihood ratio associated with the first DNA segment of the first copy of the DNA sequence. In an example, the method also includes providing the soft information to a low-density parity-check (LDPC) decoder.

Other examples describe a DNA-based storage system, comprising: a control system operable to: divide a plurality of copies of a DNA sequence into multiple DNA segments; compare corresponding DNA segments in each of the plurality of copies of the DNA sequences to determine at least one of: a ratio of DNA symbols in agreement between corresponding DNA segments of the multiple DNA segments; and a length consensus between corresponding DNA segments of the multiple DNA segments; and generate soft information associated with each of the multiple DNA segments based, at least in part, on the at least one of the ratio of DNA symbols in agreement and the length consensus. In an example, generating soft information comprises determining a bit error rate (BER) associated with each of the multiple DNA segments. In an example, the control system is further operable to generate a log likelihood ratio (LLR) associated with each of the multiple DNA segments based, at least in part, on the determined bit error rate associated with each of the multiple DNA segments. In an example, the control system is further operable to update the log likelihood ratio associated with each of the multiple DNA segments. In an example, the control system is further operable to associate a reliability bin to each of the multiple DNA segments. In an example, the control system is further operable to assign a particular log likelihood ratio (LLR) to a particular reliability bin. In an example, the control system is further operable to determine an initial log likelihood ratio in an offline environment. In an example, the control system is further operable to provide the soft information to a low-density parity-check (LDPC) decoder associated with the DNA-based storage system.

Additional examples describe a DNA-based storage system, comprising: a dense storage system storing a DNA sequence and at least one copy of the DNA sequence; and a control system operably coupled to the dense storage system and comprising: means for accessing the dense storage system to retrieve the DNA sequence and the at least one copy of the DNA sequence; means for dividing the DNA sequence and the at least one copy of the DNA sequence into corresponding DNA segments, each of the DNA segments comprising at least one DNA symbol; means for comparing the corresponding DNA segments to determine at least one of: a ratio of the DNA symbols in agreement between the corresponding DNA segments; and a length consensus between the corresponding DNA segments; and means for generating soft information associated with each of the DNA segments based, at least in part, on the at least one of the ratio of the DNA symbols in agreement and the length consensus. In an example, the means for generating soft information comprises means for determining a bit error rate (BER) associated with each of the DNA segments. In an example, the DNA-based storage system also includes means for generating a log likelihood ratio associated with the DNA segments based, at least in part, on the bit error rate associated with each of the DNA segments. In an example, the DNA-based storage system also includes means for providing the soft information to a decoding means associated with the DNA-based storage system.

The term computer-readable media as used herein may include computer storage media. Computer storage media may include volatile and nonvolatile, removable and non-removable media implemented in any method or technology for storage of information, such as computer readable instructions, data structures, or program modules. Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by a computing device (e.g., host device 505 (FIG. 5)). Any such computer storage media may be part of the computing device. Computer storage media does not include a carrier wave or other propagated or modulated data signal.

Additionally, examples described herein may be discussed in the general context of computer-executable instructions residing on some form of computer-readable storage medium, such as program modules, executed by one or more computers or other devices. By way of example, and not limitation, computer-readable storage media may comprise non-transitory computer storage media and communication media. Generally, program modules include routines, programs, objects, components, data structures, etc., that perform particular tasks or implement particular abstract data types. The functionality of the program modules may be combined or distributed as desired in various examples.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

The description and illustration of one or more aspects provided in the present disclosure are not intended to limit or restrict the scope of the disclosure in any way. The aspects, examples, and details provided in this disclosure are considered sufficient to convey possession and enable others to make and use the best mode of claimed disclosure.

The claimed disclosure should not be construed as being limited to any aspect, example, or detail provided in this disclosure. Regardless of whether shown and described in combination or separately, the various features (both structural and methodological) are intended to be selectively rearranged, included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

Aspects of the present disclosure have been described above with reference to schematic flowchart diagrams and/or schematic block diagrams of methods, apparatuses, systems, and computer program products according to embodiments of the disclosure. It will be understood that each block of the schematic flowchart diagrams and/or schematic block diagrams, and combinations of blocks in the schematic flowchart diagrams and/or schematic block diagrams, can be implemented by computer program instructions. These computer program instructions may be provided to a processor of a computer or other programmable data processing apparatus to produce a machine, such that the instructions, which execute via the processor or other programmable data processing apparatus, create means for implementing the functions and/or acts specified in the schematic flowchart diagrams and/or schematic block diagrams block or blocks. Additionally, it is contemplated that the flowcharts and/or aspects of the flowcharts may be combined and/or performed in any order.

References to an element herein using a designation such as “first,” “second,” and so forth does not generally limit the quantity or order of those elements. Rather, these designations may be used as a method of distinguishing between two or more elements or instances of an element. Thus, reference to first and second elements does not mean that only two elements may be used or that the first element precedes the second element. Additionally, unless otherwise stated, a set of elements may include one or more elements.

Terminology in the form of “at least one of A, B, or C” or “A, B, C, or any combination thereof” used in the description or the claims means “A or B or C or any combination of these elements.” For example, this terminology may include A, or B, or C, or A and B, or A and C, or A and B and C, or 2A, or 2B, or 2C, or 2A and B, and so on. As an additional example, “at least one of: A, B, or C” is intended to cover A, B, C, A-B, A-C, B-C, and A-B-C, as well as multiples of the same members. Likewise, “at least one of: A, B, and C” is intended to cover A, B, C, A-B, A-C, B-C, and A-B-C, as well as multiples of the same members.

Similarly, as used herein, a phrase referring to a list of items linked with “and/or” refers to any combination of the items. As an example, “A and/or B” is intended to cover A alone, B alone, or A and B together. As another example, “A, B and/or C” is intended to cover A alone, B alone, C alone, A and B together, A and C together, B and C together, or A, B, and C together.

What is claimed is:

1. A method for generating soft information for a DNA-based storage system, comprising:
receiving, from a DNA storage medium of the DNA-based storage system, a first copy of a DNA sequence and a second copy of the DNA sequence;
dividing the first copy of the DNA sequence into a first DNA segment and a second DNA segment, each of the first DNA segment and the second DNA segment of the first copy of the DNA sequence including at least one DNA symbol;
dividing the second copy of the DNA sequence into a first DNA segment and a second DNA segment, each of the first DNA segment and the second DNA segment of the second copy of the DNA sequence including at least one DNA symbol;
comparing the first DNA segment of the first copy of the DNA sequence to the first DNA segment of the second copy of the DNA sequence to determine first consensus information;
comparing the second DNA segment of the first copy of the DNA sequence to the second DNA segment of the second copy of the DNA sequence to determine second consensus information;
determining, based at least in part, on the first consensus information, a first bit error rate (BER) associated with the first DNA segment of the first copy of the DNA sequence; and
determining, based at least in part, on the second consensus information, a second bit error rate (BER) associated with the second DNA segment of the first copy of the DNA sequence.

2. The method of claim 1, wherein the first consensus information comprises a determination of a ratio of DNA symbols in agreement between the first DNA segment of the first copy of the DNA sequence and the first DNA segment of the second copy of the DNA sequence.

3. The method of claim 1, wherein the first consensus information comprises length information of the first DNA segment of the first copy of the DNA sequence compared to length information of the first DNA segment of the second copy of the DNA sequence.

4. The method of claim 1, further comprising associating a reliability bin with a log likelihood ratio (LLR), the log likelihood ratio being associated with the first DNA segment of the first copy of the DNA sequence.

5. The method of claim 4, wherein the log likelihood ratio associated with the first DNA segment of the first copy of the DNA sequence is determined based, at least in part, on the first bit error rate.

6. The method of claim 4, wherein the log likelihood ratio is determined in an offline environment.

7. The method of claim 4, further comprising updating the log likelihood ratio associated with the first DNA segment of the first copy of the DNA sequence.

8. The method of claim 1, further comprising providing the soft information to a low-density parity-check (LDPC) decoder.

9. A DNA-based storage system, comprising:
a control system operable to:
divide a plurality of copies of a DNA sequence into multiple DNA segments;
compare corresponding DNA segments in each of the plurality of copies of the DNA sequences to determine at least one of:
a ratio of DNA symbols in agreement between corresponding DNA segments of the multiple DNA segments; and
a length consensus between corresponding DNA segments of the multiple DNA segments; and
generate soft information associated with each of the multiple DNA segments based, at least in part, on the at least one of the ratio of DNA symbols in agreement and the length consensus.

10. The DNA-based storage system of claim 9, wherein generating soft information comprises determining a bit error rate (BER) associated with each of the multiple DNA segments.

11. The DNA-based storage system of claim 10, wherein the control system is further operable to generate a log likelihood ratio (LLR) associated with each of the multiple DNA segments based, at least in part, on the determined bit error rate associated with each of the multiple DNA segments.

12. The DNA-based storage system of claim 11, wherein the control system is further operable to update the log likelihood ratio associated with each of the multiple DNA segments.

13. The DNA-based storage system of claim 10, wherein the control system is further operable to associate a reliability bin to each of the multiple DNA segments.

14. The DNA-based storage system of claim 13, wherein the control system is further operable to assign a particular log likelihood ratio (LLR) to a particular reliability bin.

15. The DNA-based storage system of claim 10, wherein the control system is further operable to determine an initial log likelihood ratio in an offline environment.

16. The DNA-based storage system of claim 10, wherein the control system is further operable to provide the soft information to a low-density parity-check (LDPC) decoder associated with the DNA-based storage system.

17. A DNA-based storage system, comprising:
a dense storage system storing a DNA sequence and at least one copy of the DNA sequence; and
a control system operably coupled to the dense storage system and comprising:
means for accessing the dense storage system to retrieve the DNA sequence and the at least one copy of the DNA sequence;
means for dividing the DNA sequence and the at least one copy of the DNA sequence into corresponding DNA segments, each of the DNA segments comprising at least one DNA symbol;
means for comparing the corresponding DNA segments to determine at least one of:
a ratio of the DNA symbols in agreement between the corresponding DNA segments; and
a length consensus between the corresponding DNA segments; and
means for generating soft information associated with each of the DNA segments based, at least in part, on the at least one of the ratio of the DNA symbols in agreement and the length consensus.

18. The DNA-based storage system of claim 17, wherein the means for generating soft information comprises means for determining a bit error rate (BER) associated with each of the DNA segments.

19. The DNA-based storage system of claim 18, further comprising means for generating a log likelihood ratio associated with the DNA segments based, at least in part, on the bit error rate associated with each of the DNA segments.

20. The DNA-based storage system of claim 17, further comprising means for providing the soft information to a decoding means associated with the DNA-based storage system.
